(19) United States
(12) Patent Application Publication
(10) Pub. No.: US 2023/0270812 A1
(43) Pub. Date: Aug. 31, 2023

(54) COMPOSITION TO BE USED FOR STRENGTHENING THE IMMUNE SYSTEM IN HUMAN AND ANIMAL TO ACQUIRE PAN-IMMUNITY

(71) Applicant: LEGACY HEALTHCARE (SWITZERLAND) SA, Epalinges (CH)

(72) Inventors: Saad HARTI, Epalinges (CH); Jiawei LIU, Geneve (CH)

(73) Assignee: LEGACY HEALTHCARE (SWITZERLAND) SA, Epalinges (CH)

(21) Appl. No.: 17/922,884

(22) PCT Filed: May 7, 2021
(86) PCT No.: PCT/EP2021/062135

§ 371 Date: Nov. 2, 2022

(30) Foreign Application Priority Data
May 7, 2020 (FR) FR2004533

(51) Int. Cl. A61K 36/752; A61K 36/77; A61K 36/8962; A61K 9/14; A61P 37/02; A61P 29/00

(52) U.S. Cl. CPC A61K 36/752; A61K 36/77; A61K 36/8962; A61K 9/14; A61P 37/02; A61P 29/00

(57) ABSTRACT

The disclosure concerns a composition for strengthening the immunity system to acquire pan-immunity, for the prevention and treatments of infectious and other diseases with immunological components, including eliminating cancer cells. The administration of the composition can be by oral, parenteral or non-parenteral routes, local injection, topical application, or in combinations thereof, and the administration of the composition as adjuvant potentiator in various therapies/treatments uses the aforenamed routes. The composition contains, as active ingredients, an extract of Allium species and an extract of Citrus species and an extract of Paullinia species and an extract of Theobroma species.

### CROSS-REFERENCE TO RELATED APPLICATIONS

The present application is a national phase entry of PCT application serial no. PCT/EP2021/062135 filed on May 7, 2021, which claims priority to French application serial no. 2004533 filed May 7, 2020, both of which are incorporated by reference herein.

### TECHNICAL FIELD

The invention concerns compositions used in a method for strengthening the immune system of human and animal to acquire pan-immunity.

### BACKGROUND

Facing the wide variety of disease-causing pathogens, the immune system is essential to our survival. Immunological impairment has devastating consequences. Good and healthy immune system orchestrates a potent defense that consists of affecting the functions of immune cells or affecting antibody secretion to control the infection to maintain immune homeostasis. This is possible through direct or indirect involvement of molecular and cellular inflammatory processes. It is therefore of great importance to enhance the immune system's protective actions against pathogenic agents and tumor cells and to control its actions in order not to develop autoimmune diseases and other disorders with immunological components.

The invention concerns a composition used in a method for strengthening the immune systems of human and animal to acquire pan-immunity. The purpose of the method is to treat and prevent all types of pathogen infection (e.g. bacteria, viruses, fungi, prion and ectoparasites, as well as protozoan infection), and all immune-related diseases that are due to dysfunction of immune system, including eliminating cancer cells. The diseases can be spread through, but not limited to skin contact, the transfer of bodily fluids, contact with faces, ingesting contaminated food or water, inhaling airborne particles or droplets, touching an object carrying the pathogen, etc. Particularly, the method deals with all symptoms/syndromes related the above-mentioned conditions, as well as the post-infection and post-treatment symptoms/syndromes, such as Post-Treatment Lyme Disease Syndrome.

Sometimes there is no visible symptom following infection. Some examples of the symptoms following infection include, but not limited to: fever, chills, cold-related symptoms (e.g. runny nose, sneezing, and congestion), Influenza-related symptoms (e.g. muscle pain, cough, congestion, headache, fatigue), strep throat symptoms (e.g. sore throat, swollen lymph nodes), urinary tract infection symptoms (e.g. pelvic pain, urinating pain and blood in the urine, back pain due to kidney infection), infectious cellulitis (e.g. swollen red and sensitive ski), gastroenteritis (e.g. diarrheal, cramps, vomiting), acute pneumonia (e.g. cough with expectoration of phlegm or pus), severe acute respiratory syndrome, otitis media (e.g. ear pain and fluid leaking out of ear or hearing loss), sexually transmitted diseases, etc.

Our study showed that a composition comprising the ingredients mentioned hereafter enabled significant improvement of health condition related to strengthened and good function of immune system. However, the present invention is a method to improve the general immunologic status, which is different from an antibiotic invention, which needs test against certain pathogens. The composition in the present invention has a function to ameliorate immune system as revealed at two scientifically proved levels: 1. increased volunteer wheel running activity (VWRA); and 2. down-regulation of il-8, an indirect method to measure immune-improving or immunomodulatory bio-activity (see descriptions below).

Based on state-of-the-art scientific knowledge known to those skilled in the art, the experimental data in the present application show evidence that the composition has beneficial effects on immunologic status. Such beneficial effects are shown by the capacity of “voluntary wheel-running” in the mouse model, as illustrated hereafter.

Scientific evidence exists showing an association between improved immunity and increased voluntary wheel running activity in rodent (as illustrated below).

Earlier studies pinpointed numerous genes expression patterns annotated to the immune process, immune response, and immune system organ morphology, as well as cytokine interactions were differentially expressed between high voluntary running capacity mice and control mice (Zhang et al. Brain region-dependent gene networks associated with selective breeding for increased voluntary wheel-running behavior. PLOS ONE, Aug. 2, 2018 https://doi.org/10.1371/journal.pone.0201773).

Other studies demonstrated that intake of certain natural immunity-improving components, such as those found in fermented soymilk (FSM) increased voluntary wheel running activity in rodent. Further analyses revealed that following FSM administration, there was an increased protein production of tyrosine hydroxylase (TH), suggesting the involvement of dopamine signaling. Indeed, tyrosine hydroxylase is key for dopamine production because dopamine is derived from a two-step process starting with the hydroxylation of L-tyrosine by the enzyme TH (Sato et al. (2010), Fermented soymilk increases voluntary wheel running activity and sexual behavior in male rats. Appl. Physiol. Nutr. Metab. 35: 749-754; Wagar et al. (2009), Immunomodulatory Properties of Fermented Soy and Dairy Milks Prepared with Lactic Acid Bacteria. JOURNAL OF FOOD SCIENCE, Vol. 74, Nr. 8).

Dopamine is well recognized as a neurotransmitter in the brain. It regulates, as a broad immunomodulator, critical functions in a variety of peripheral systems, particularly regulating immune function. Many immune cells (e.g. T-lymphocytes) express dopamine receptors, enabling them to actively respond centrally and peripherally to dopamine. Hence, dopaminergic immunoregulation is an important part of immune function. Furthermore, the bone marrow microenvironment is critical in the maintenance of hematopoietic stem cells (HSCs), from which immune cells are derived through hematopoiesis. Dopamine signaling has been shown to regulate HSCs development and function. Meanwhile, dopamine regulates a variety of immune functions including cytokine secretion (Matt et al. Where Is Dopamine and how do Immune Cells See it?:Dopamine-Mediated Immune Cell Function in Health and Disease. Journal of Neuroimmune Pharmacology, 11 May 2019).

Immunomodulatory bioactivity of a variety of bioactive components (e.g. those found in fermented soy beverage) can be assessed on the pro-inflammatory response of intestinal epithelial cells (IEC) following Tumor Necrosis Factor α (TNFα) treatment, by measuring the impact on IL-8 production. Regarding the immunity-improving fermented soy beverage and milk blend, down-regulation of IL-8 production has been observed (Wagar et al above), finding consistent with the observations for the immunity-improving bioactivity of the composition according to the present application, i.e. down-regulation of IL-8 production in TNFα-challenged human endothelial cells (see below “Example 2: Immunomodulatory/anti-inflammatory effect”).

On the other hand, the immunomodulatory effect of the composition according to the invention is shown in the immunosuppressed mouse model by increased voluntary wheel running capacity.

The animal model used in the application is a mouse model of fatigue induced by peripheral irradiation. It has been documented that irradiation can cause generalized immunosuppression, mainly due to lymphocyte apoptosis and dysfunction of immune regulation (McFarland et al. (2012), Regulatory T Cells in γ Irradiation-Induced Immune Suppression. PLoS ONE 7(6): e39092. doi:10.1371/journal.pone.0039092).

Under such circumstances, without treatment, as shown by the experimental results in the present application (see Table 1 and FIG. 1) the Voluntary Wheel Running Activity (VWRA) of mice was greatly reduced. However, the intake of the composition according to the invention improved such a wheel running capacity (both running distance and speed, see Table 1 and FIGS. 2, 3, 4) post-irradiation, showing an amelioration of immunologic status, which is associated, as explained above, with the increased voluntary wheel running capacity of mice. The study outcome summary is presented hereafter in the Experimental Part in Table 1 and in FIGS. 1-6.

Thus, a composition according to the invention, comprising the ingredients mentioned hereafter, provides with a method for strengthening the immune system for good immune functions and finally acquire pan-immunity.

A composition comprising the ingredients mentioned hereafter was administered orally to mice with immune deficiency conditions, in a placebo-controlled experiment. It was observed that, compared to placebo-treated mice, the composition significantly improved their health status thanks to reestablishment of good function of the strengthened immune system, demonstrated and measured by significantly increased wheel running capacity.

Activation of the immune system by infection, injury, or trauma leads to the release of pro-inflammatory cytokines and other immune factors, in some cases, even resulting in “cytokine storm” (a dangerous overreaction of the immune system). These cytokines orchestrate local and systemic riotous immune responses and mediate various pathologic conditions.

A study of the composition according to the invention, on its immunity-improving/immunomodulatory/anti-inflammatory effect via down-regulation of TNF alpha-induced expression of pro-inflammatory molecules: Interleukin 8, ICAM-1 and E-selectin, is also presented hereafter in the Experimental Part in Tables 2-4.

### SUMMARY

The present invention concerns a composition to be used for strengthening the immunity system containing as active ingredient an extract of Allium species, an extract of Citrus species and an extract of Paullinia species and an extract of Theobroma species. The extract of Paullinia species and/or the extract of Theobroma species can be atomized or not.

According to another embodiment of the present invention, the said composition contains from 20% to 80% by weight of an extract of Allium species, from 1.5% to 20% by weight of an extract of Citrus species, from 0.05% to 5.0% by weight of an extract of Paullinia species and from 0.05% to 5.0% by weight of an extract of Theobroma species, based on the total weight of the four active ingredients.

According to a further embodiment of the present invention, the said composition contains from 60% to 70% by weight of an extract of Allium cepa, from 7% to 12% by weight of an extract of Citrus limon, from 0.08% to 0.20% by weight of an extract of Paullinia cupana and from 0.06% to 0.18% by weight of an extract of Theobroma cacao, based on the total weight of the four active ingredients.

Also, according to an embodiment of the present invention, the said composition contains as excipients from 1.0% to 8.0% by weight of sodium chloride and from 10% to 40% by weight of glycerine, based on the total weight of the composition. This composition is to be used to acquire pan-immunity.

According to another embodiment, the composition is to be used to prevent and to treat infectious and other diseases with immunological components, such as viral infections and/or auto-immune diseases, including eliminating cancer cells.

Also, according to a further embodiment, this composition is to be used as adjuvant potentiator in various therapies or treatments. This composition is to be administrated by parenteral route, non-parenteral route, oral route or local or topical routes, or in combination thereof.

### BRIEF DESCRIPTION OF THE DRAWINGS

FIG. 1: VWRA of placebo-mice was greatly reduced, and reached the lowest running distance at day 3 post-irradiation. Mice recovered gradually over the next several days, returning to the initial running distance measured in the pre-irradiation period.

FIG. 2: Average cumulative distance run during post-irradiation by “the composition” group (“Drug”) was 17% greater than Placebo group. “The composition” group mice returned to daily running distance baseline after 7 days; placebo mice returned to baseline running distance 12 days after irradiation.

FIG. 3: As in the described model, a decrease in voluntary wheel speed is most pronounced during the late hours of the active phase (i.e. dark cycle). The average post-irradiation running speed was systematically greater in the “the composition” group (“Drug”) versus placebo group, in every section of the active phase.

FIG. 4: As in the described model, a decrease in voluntary wheel distance is most pronounced during the late hours of the active phase (i.e. dark cycle). The average post-irradiation running distance was systematically greater in the “the composition” group (“Drug”) versus placebo group, in every section of the active phase.

FIG. 5: Both “the composition” and Placebo were given in a 1% saccharin solution. Volume of solution consumed by mice in “the composition” group (“Drug”) was less than mice in Placebo group, before and after irradiation.

FIG. 6: The mean group body weight showed that there was no difference in body weight between mice in Placebo group and mice in “the composition” group (“Drug”).

### DETAILED DESCRIPTION

It has been discovered that the administration by oral route, or parenteral route, or local injection, or a combination thereof, of a composition containing as active ingredient an extract of Allium species, an extract of Citrus species and an extract of Paullinia species and an extract of Theobroma species, has a novel and previously unknown effect for strengthening the immune system in human and animal in order to acquire pan-immunity.

A composition comprising the ingredients mentioned hereafter was administered topically to human subjects with abnormal conditions in terms of inflammation. It was observed that, the composition significantly improved the inflammatory skin condition thanks to the anti-inflammatory function of the composition.

The present invention proposes a method for the improvement/strengthening of the immune system; comprising the administration by oral, parenteral, topical application, or local injection, and/or as adjuvant potentiator in various therapies/treatments, or in combination, of a composition containing as active ingredient an extract of Allium species, an extract of Citrus species and an extract of Paullinia species and an extract of Theobroma species.

In particular, the present invention concerns a method for strengthening immunity system; comprising the administration by oral route, or parenteral route, or topical application, or injection, or a combination thereof, of a composition containing as active ingredient an extract of Allium species, an extract of Citrus species and an extract of Paullinia species and an extract of Theobroma species. Among the methods for strengthening immunity system to acquire pan-immunity, according to the invention, those which are of more particular interest are the methods in which the preferred composition contains from 20% to 80% by weight of an extract of Allium species, from 1.5% to 20% by weight of an extract of Citrus species, from 0.05% to 5.0% by weight of an extract (atomized or not) of Paullinia species and from 0.05% to 5.0% by weight of an extract (atomized or not) of Theobroma species, based on the total weight of the four active ingredients. According to an embodiment, the composition comprises from 20% to 80% by weight of an extract of Allium species, from 1.5% to 20% by weight of an extract of Citrus species, from 0.05% to 5.0% by weight of an extract (atomized or not) of Paullinia species, from 0.05% to 5.0% by weight of an extract (atomized or not) of Theobroma species, from 1.0% to 8.0% by weight of Sodium chloride and from 10% to 40% by weight of glycerine, based on the total weight of the composition.

Among the methods for strengthening the immunity system, those which are the more preferred interest are the methods in which the compositions are used not only as conventional oral composition, composition for injection (local or systemic), but also as an adjuvant potentiator in therapies/treatments for strengthening the immunity system to acquire pan-immunity. The compositions contain from 20% to 80% by weight of an extract of Allium species, from 1.5% to 20% by weight of an extract of Citrus species, from 0.05% to 5.0% by weight of an extract (atomized or not) of Paullinia species and from 0.05% to 5.0% by weight of an extract (atomized or not) of Theobroma species, based on the weight of the four active ingredients.

According to an embodiment, the compositions contain from 20% to 80% by weight of an extract of Allium species, from 1.5% to 20% by weight of an extract of Citrus species, from 0.05% to 5.0% by weight of an extract (atomized or not) of Paullinia species and from 0.05% to 5.0% by weight of an extract (atomized or not) of Theobroma species, based on the total weight of the four active ingredients. According to an embodiment, the composition comprises from 20% to 80% by weight of an extract of Allium species, from 1.5% to 20% by weight of an extract of Citrus species, from 0.05% to 5.0% by weight of an extract (atomized or not) of Paullinia species, from 0.05% to 5.0% by weight of an extract (atomized or not) of Theobroma species, from 1.0% to 8.0% by weight of Sodium chloride and from 10% to 40% by weight of glycerine, based on the total weight of the composition.

The term extract of Allium species refers particularly to extracts and native extracts obtained from all species of the genus Allium (family Liliaceae), and especially Allium cepa.

The term genus citrus refers particularly to the family Rutaceae, especially Citrus limon.

The term extract (atomized or not) of Paullinia species refers particularly to extracts and native extracts obtained from all species of the genus Paullinia (family Sapindaceae), especially Paullinia cupana (Guarana).

The term extract (atomized or not) of Theobroma species refers particularly to aqueous-alcoholic extracts and native extracts obtained from all species of the genus Theobroma (family Malvaceae), and especially Theobroma cacao.

The most preferred compositions used according to the invention are: those containing approximately 66% by weight of an extract of Allium cepa, approximately 9% by weight of an extract of Citrus limon, approximately 0.11% by weight of an extract (atomized or not) of Paullinia cupana and approximately 0.11% by weight of an extract (atomized or not) of Theobroma cacao, based on the total weight of the four active ingredients. According to the invention, the composition is chronically administered in a mixture containing as active ingredient an extract of Allium species, an extract of Citrus species and an extract of Paullinia species and an extract of Theobroma species. According to an embodiment of the invention the composition is administered daily during a period of several months or longer with a composition containing as active ingredients an extract of Allium species, an extract of Citrus species and an extract of Paullinia species and an extract of Theobroma species.

In order to obtain a measurable effect on the strengthening of the immunity system to acquire pan-immunity, it is necessary to perform the administration of the compositions chronically. When using the compositions obtained according to the invention, doses may vary within relatively wide limits and must be set according to the person being treated and the condition concerned. Pharmaceutical compositions normally contain from 0.4 to 1000 mg, preferably from 2 to 400 mg, of active ingredients as defined above, in the form of dry extract or liquid solution. For example, the composition can be orally administered in human, twice daily, with 5 ml each intake.

The present invention also concerns a composition containing as active ingredient an extract of Allium species, an extract of Citrus species and an extract of Paullinia species and an extract of Theobroma species for use in strengthening the immunity system in order to acquire pan-immunity, by oral, parenteral, other above-mentioned routes, and/or as adjuvant potentiator in various therapies/treatments, or in combination thereof. According to an embodiment of the invention the composition for use in strengthening the immunity system in order to acquire pan-immunity contains an extract of Allium species, an extract of Citrus species and an extract (atomized or not) of Paullinia species and an extract (atomized or not) of Theobroma species. According to a further embodiment of the invention the composition for use in strengthening the immunity system in order to acquire pan-immunity, contains from 20% to 80% by weight of an extract of Allium species, from 1.5% to 20% by weight of an extract of Citrus species, from 0.05% to 5.0% by weight of an extract (atomized or not) of Paullinia species and from 0.05% to 5.0% by weight of an extract (atomized or not) of Theobroma species, based on the total weight of the four active ingredients.

### EXAMPLES OF COMPOSITIONS/TREATMENTS

### Example 1

The mice have received, every day by oral route, a treatment with a composition having, independently from the excipients, the following composition:
an extract of Allium cepa (containing quercetin): 71%
an extract of Citrus limon: 1.7%
an atomised extract of Paullinia cupana: 4.5%
an atomised extract of Theobroma cacao: 4.8%

### Example 2

The mice have received, every day by oral route, a treatment with a composition having, independently from the excipients, the following composition:
an extract of Allium cepa (containing quercetin): 60.50%
an extract of Citrus limon: 5.25%
an atomised extract of Paullinia cupana: 2.5%
an atomised extract of Theobroma cacao: 2.3%

### Example 3

The mice have received, every day by oral route, a treatment with a composition having, independently from the excipients, the following composition:
an extract of Allium cepa (containing quercetin): 52.50%
an extract of Citrus limon: 6.20%
an atomised extract of Paullinia cupana: 1%
an atomised extract of Theobroma cacao: 1%

### Example 4

The mice have received, every day by oral route, a treatment with a composition having, independently from the excipients, the following composition:
an extract of Allium cepa (containing quercetin): 48.50%
an extract of Citrus limon: 20.5%
an atomised extract of Paullinia cupana: 1.5%
an atomised extract of Theobroma cacao: 1.35%

### Example 5

The mice have received, every day by oral route, a treatment with a composition having, independently from the excipients, the following composition:
an extract of Allium cepa (containing quercetin): 45.50%
an extract of Citrus limon: 20%
an atomised extract of pulverized Paullinia cupana: 0.75%
an atomised extract of Theobroma cacao: 0.65%

### Example 6

The mice have received, every day by oral route, a treatment with a composition having, independently from the excipients, the following composition:
an extract of Allium cepa (containing quercetin): 31.50%
an extract of Citrus limon: 18%
an atomised extract of Paullinia cupana: 0.15%
an atomised extract of pulverized Theobroma cacao: 0.18%

### Example 7

The mice have received, every day by oral route, a treatment with a composition having, independently from the excipients, the following composition:
an extract of a species of the genus Allium belonging to the family Liliaceae other than Allium cepa: 30.50%
an extract of Citrus limon: 3.20%
an extract of Paullinia cupana: 0.06%
an atomised extract of Theobroma cacao: 0.05%

### Example 8

The mice have received, every day by oral route, a treatment with a composition having, independently from the excipients, the following composition:
an extract of Allium cepa: 30.50%
an extract of Citrus limon: 3.20%
an atomised extract of Paullinia cupana: 0.06%
an extract of Theobroma cacao: 0.05%

### Example 9

The mice have received, every day by oral route, a treatment with a composition having, independently from the excipients, the following composition:
an extract of Allium cepa: 35.50%
an extract of Citrus limon: 3.80%
an atomised extract of Paullinia cupana: 0.03%
an atomised extract of Theobroma cacao: 0.03%

### Example 10

The mice have received, every day by oral route, a treatment with a composition having, independently from the excipients, the following composition:
an extract of Allium cepa 36.45%
an extract of Citrus limon: 10%
an extract of Paullinia cupana: 2.5%
an hydroalcoholic extract of Theobroma cacao : 1.35%
   The excipients used in the above examples are from 1.0% to 8.0% by weight of sodium chloride and from 10% to 40% by weigh of glycerine, based on the total weight of the composition.

### EXPERIMENTAL RESULTS

The results are obtained by using the above compositions within the ranges in the examples 1 to 10. For all the above compositions, the tests performed on mice have surprisingly shown a strengthening of their immunity system significantly demonstrated according to a mode of evidence known in the art, as explained above, measured by their increased wheel running capacity in comparison with the level of their immunity system before intake of the composition.

This indicates that the composition according to the present invention is capable of positively influencing and strengthening the immunity system.

Our studies have shown that a composition comprising the ingredients mentioned hereafter enabled significant improvement of health conditions related to strengthened and good function of immune system.

### Example 1: Wheel Running Activity

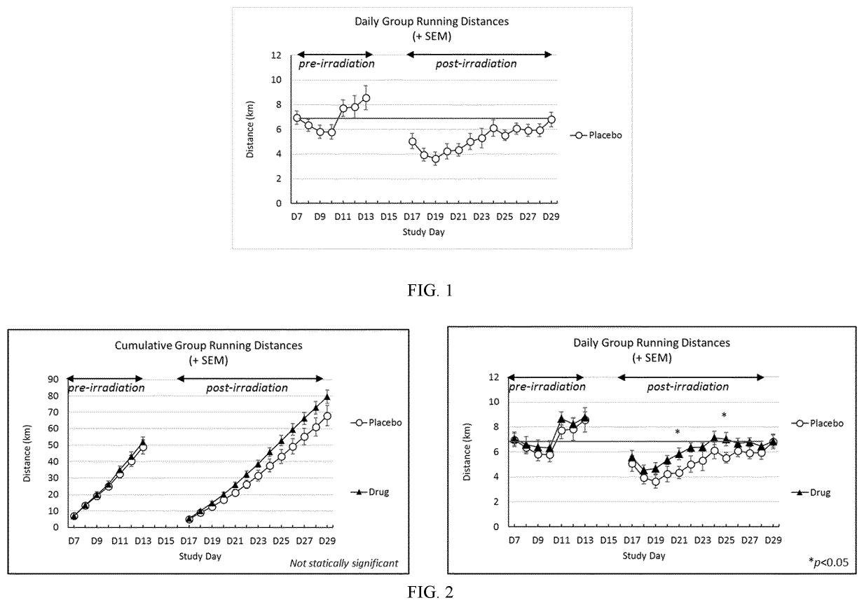

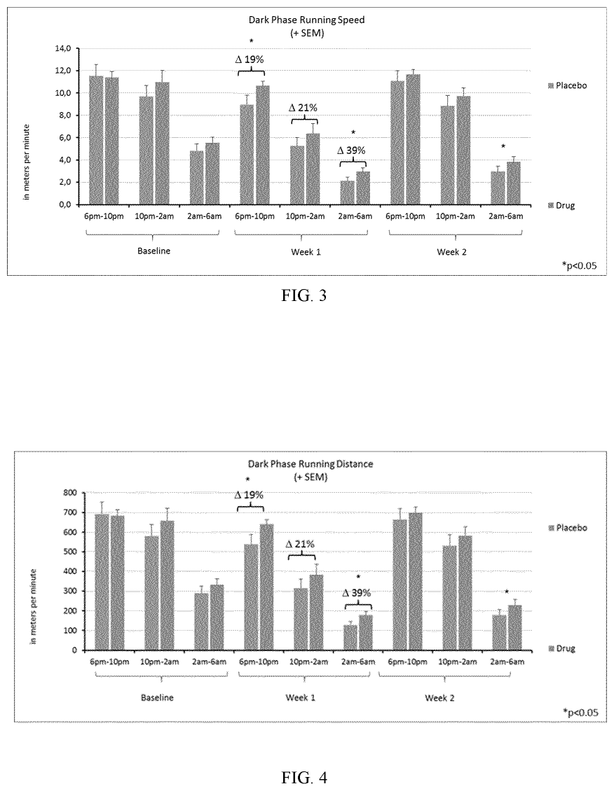

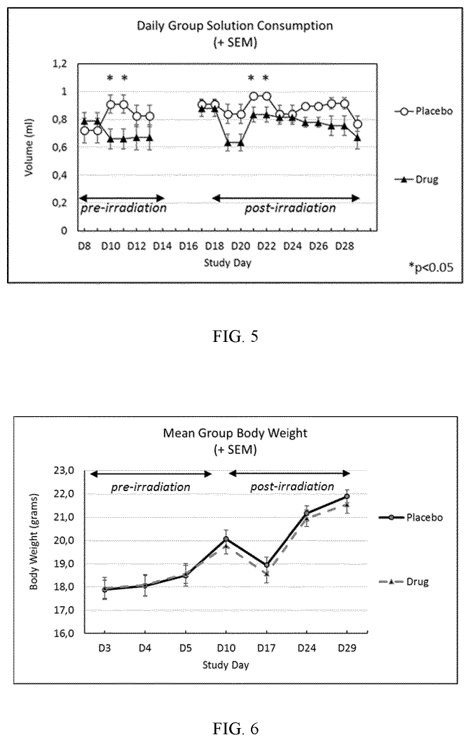

The study outcome summary is presented below in Table 1 and FIGS. 1-6.

TABLE 1
Outcome of study on the composition to improve immunologic
status in a mouse model, that is shown by enhanced
Voluntary Wheel Running Activity (VWRA)

| FIG. | Topic | Study on “the composition” (see FIG.) |
|---|---|---|
| | Experimental | 20 mice irradiated (800 cGy) |
| | setting | 10 taking “the composition” |
| | | 10 taking Placebo |
| 1 | Irradiation | VWRA of Placebo mice reached the lowest |
| | and VWRA | amount of VWRA 3 days post-irradiation. Mice |
| | | recovered gradually over the next several days, |
| | | until returned to baseline running distance of pre- |
| | | irradiation period. |
| 2 | Effect of “the | Average cumulative distance run during post- |
| | composition” | irradiation by “the composition” group was 17% |
| | on VWRA | greater than Placebo group. |
| | after | Statistically significant difference in several time- |
| | irradiation | points. |
| | | “The composition” group mice returned to daily |
| | | running baseline distance after 7 days. |
| | | Placebo mice returned to baseline running |
| | | distance 12 days after irradiation. |
| 3 | Speed by 4 | As previously observed in the model, a decrease |
| | hours | in voluntary wheel running speed was most |
| | increment | pronounced during the late hours of the active |
| | during of the | phase (i.e. dark cycle). |
| | active phase | The post-irradiation running speed was |
| | (dark cycle) | systematically greater in “the composition” group |
| | | versus Placebo group, in every section of the |
| | | active phase. |
| | | Statistically significant difference in several time- |
| | | points. |
| 4 | Distance by | Distance was systematically greater in “the |
| | 4 hours | composition” group versus Placebo group, in |
| | increment | every section of the active phase. |
| | during of the | Statistically significant difference in several time- |
| | active phase | points. |
| | (dark cycle) | As observed in the model, decreases in voluntary |
| | | wheel running distance were most pronounced |
| | | during the late hours of the active phase. |
| 5 | Solution | Volume of solution consumed by mice in “the |
| | consumption | composition” group was less than mice in Placebo |
| | | group, before and after irradiation. |
| | | Statistically significant difference in several time- |
| | | points. |
| 6 | Body weight | There was no difference in body weight between |
| | | mice in Placebo group and mice in “the |
| | | composition” group. |
| | Other | No overt co-morbidities, such as anemia, |
| | physiological | anhedonia (assessed by saccharine preference |
| | &behavioral | testing), change in weight, etc., were observed. |
| | observation | |

### Example 2: Immunity-Improving/Immunomodulatory/Anti-Inflammatory Effect

The study outcome summary is presented below in Tables 2-4. The composition within the ranges in the examples 1 to 10, containing as active ingredient an extract of Allium species, an extract of Citrus species and an extract of Paullinia species and an extract of Theobroma species, was used to perform the study on its immunity-improving/immunomodulatory/anti-inflammatory effect via down-regulation of TNF alpha-induced expression of pro-inflammatory molecules: Interleukin-8, ICAM-1 and E-selectin, as summarized in Table 2-4.

TABLE 2
The effects of “composition” on the expression of cytokine
Interleukin-8 (IL-8) by endothelial cells (HUVECs)

| | Non-treated | TNF-treated | % Increase (+) |
|---|---|---|---|
| | cells | cells | % Reduction (−) |
| | measured by | measured by | compared to |
| Composition | sABC* | sABC* | control |
| Medium (control) | 1 164 | 5 406 | |
| Composition | 887 | 4 827 | −11% |

*specific Antibody Bound per Cell

TABLE 3
The effects of “composition” on the expression
of adhesion molecule E-selectin/ELAM-1 (CD62E) on the
surface of endothelial cells (HUVECs)

| | Non-treated | TNF-treated | % Increase (+) |
|---|---|---|---|
| | cells | cells | % Reduction (−) |
| | measured by | measured by | compared to |
| | sABC* | sABC* | control |
| Medium (control) | Non detectable | 3 074 | |
| Composition | 65 | 1 648 | −46% |

*specific Antibody Bound per Cell

TABLE 4
The effects of “composition” on the expression of adhesion molecule
ICAM-1 (CD54) on the surface of endothelial cells (HUVECs)

| | | TNF-treated | % Increase (+) |
|---|---|---|---|
| | Non-treated cells | cells | % Reduction (−) |
| | measured by | measured by | compared to |
| | sABC* | sABC* | control |
| Medium (control) | 3 172 | 386 181 | |
| Composition | 1 664 | 254 171 | −34% |

*specific Antibody Bound per Cell

Hence, the above study shows that a composition according to the invention enabled modulation of the over-reaction of immune response through anti-inflammation. Consequently, the composition improved health condition as a result of good functioning of the strengthened immune system. Thus, a composition according to the invention provides a method for strengthening the immune system for good immune functions and finally acquire pan-immunity.

The invention claimed is:

1. A composition to be used for strengthening the immunity system in a human subject or an animal subject, the composition comprising as active ingredients an extract of Allium species and an extract of Citrus species and an extract of Paullinia species and an extract of Theobroma species.

2. A composition for strengthening the immunity system according to claim 1, wherein the extract of Paullinia species and/or the extract of Theobroma species are atomized.

3. A composition for strengthening the immunity system according to claim 1, said wherein the composition comprises from 20% to 80% by weight of the extract of Allium species, from 1.5% to 20% by weight of the extract of Citrus species, from 0.05% to 5.0% by weight of the extract of Paullinia species, and from 0.05% to 5.0% by weight of the extract of Theobroma species based on the total weight of the four active ingredients.

4. A composition for strengthening the immunity system according to claim 1, said wherein the extract of Allium species comprises an extract of Allium cepa, the extract of Citrus species comprises an extract of Citrus limon, the extract of Paullinia species comprises an extract of Paullinia cupana, and the extract of Therobroma species comprises an extract of Therobroma cacao, and wherein the composition comprises from 60% to 70% by weight of the extract of Allium cepa, from 7% to 12% by weight of the extract of Citrus limon, from 0.08% to 0.20% by weight of the extract of Paullinia cupana, and from 0.06% to 0.18% by weight of the extract of Theobroma cacao based on the total weight of the four active ingredients.

5. A composition for strengthening the immunity system according to claim 1, wherein the extract of Allium species comprises an extract of Allium cepa, the extract of Citrus species comprises an extract of Citrus limon, the extract of Paullinia species comprises an extract of Paullinia cupana, and the extract of Therobroma species comprises an extract of Therobroma cacao, and wherein the composition comprises from 32% to 45% by weight of the extract of Allium cepa, 6% to 12% of the extract of Citrus limon, 2% to 3% of the extract of Paullinia cupana, 0.25% to 3.2% of an hydroalcoholic extract of the extract of Theobroma cacao.

6. A composition for strengthening the immunity system according to claim 1, wherein the composition further comprises as excipients from 1.0% to 8.0% by weight of sodium chloride and from 10% to 40% by weight of glycerine based on the total weight of the composition.

7. A composition for strengthening the immunity system according to claim 1, to be used to acquire pan-immunity.

8. A composition for strengthening the immunity system according to claim 1, to be used to acquire pan-immunity and to prevent and to treat infectious and other immune-related diseases that are due to dysfunction of immune system including eliminating cancer cells.

9. A composition for strengthening the immunity system according to claim 1, to be used as an adjuvant potentiator in various therapies or treatments.

10. A composition for strengthening the immunity system according to claim 1, in an appropriate form to be administrated by parenteral, non-parenteral, oral, local, topical routes, or in combinations thereof.

11. A supplemental immunity composition comprising:
greater than or equal to about 20 wt. % to less than or equal to about 80 wt. % of an Allium species extract;
greater than or equal to about 1.5 wt. % to less than or equal to about 20 wt. % of a Citrus species extract;
greater than or equal to about 0.05 wt. % to less than or equal to about 5 wt. % of a Paullinia species extract;
greater than or equal to about 0.05 wt. % to less than or equal to about 5 wt. % of a Theobroma species extract;
greater than or equal to about 1 wt. % to less than or equal to about 8 wt. % of sodium chloride; and
greater than or equal to about 10 wt. % to less than or equal to about 40 wt. % of glycerine.

12. The supplemental immunity composition of claim 11, wherein the Allium species extract comprises an Allium cepa extract.

13. The supplemental immunity composition of claim 11, wherein the Citrus species extract comprises a Citrus limon extract.

14. The supplemental immunity composition of claim 11, wherein the Paullinia species extract comprises a Paullinia cupana extract.

15. The supplemental immunity composition of claim 11, wherein the Theobroma species extract comprises a Theobroma cacao extract.

16. The supplemental immunity composition of claim 11, wherein at least one of the Paullinia species extract and the Theobroma species extract is atomized.

17. A method for strengthening the immunity system in subject, the method comprising:
administering to the subject a supplemental immunity composition comprising:
an extract of Allium species;
an extract of Citrus species;
an extract of Paullinia species; and
an extract of Theobroma species.

18. The method of claim 17, wherein the supplemental immunity composition comprises:
greater than or equal to about 20 wt. % to less than or equal to about 80 wt. % of the Allium species extract;
greater than or equal to about 1.5 wt. % to less than or equal to about 20 wt. % of the Citrus species extract;
greater than or equal to about 0.05 wt. % to less than or equal to about 5 wt. % of the Paullinia species extract; and
greater than or equal to about 0.05 wt. % to less than or equal to about 5 wt. % of the Theobroma species extract.

19. The method of claim 17, wherein the supplemental immunity composition further comprises:
greater than or equal to about 1 wt. % to less than or equal to about 8 wt. % of sodium chloride; and
greater than or equal to about 10 wt. % to less than or equal to about 40 wt. % of glycerine.

20. The method of claim 17, wherein the Allium species extract comprises an Allium cepa extract, the Citrus species extract comprises a Citrus limon extract, the Paullinia species extract comprises a Paullinia cupana extract, and the Theobroma species extract comprises a Theobroma cacao extract.
